(19) United States
(12) Patent Application Publication
(10) Pub. No.: US 2003/0093084 A1
(43) Pub. Date: May 15, 2003

(54) Delivery devices for flow regulating implants

(75) Inventors: Oded Nissan, Modiin (IL); Sagi Ben-Rimon, Jerusalem (IL); Ira Yaron, Har Adar (IL)

Correspondence Address: KENYON & KENYON, ONE BROADWAY, NEW YORK, NY, 10004, US

(73) Assignee: OPTONOL LTD., Neve Ilan (IL)

(21) Appl. No.: 09/987,134

(22) Filed: Nov. 13, 2001

(51) Int. Cl. A61F 11/00

(52) U.S. Cl. 606/108

(57) ABSTRACT

The disclosed delivery devices for flow regulating implants comprise a handle and a tube (such as a hollow needle or cannula), with a retractable wire extending through the tube. Prior to deployment, the wire extends a short distance out of the distal end of the tube. The flow regulating implant is mounted on the wire at the distal end of the tube; for example, the implant may have a central bore into which the wire extends. The implant may be held on the wire by friction, by a hook, or by other suitable means. The delivery device includes a trigger that acts on the retractable wire. Actuating the trigger retracts the wire into the tube, thereby releasing the implant.

### FIELD OF THE INVENTION

The invention relates generally to delivery devices for delivering and/or implanting flow regulating implants, which are medical implants used to regulate the flow of fluids within the body. The delivery devices may be used, for example, to deliver and/or implant ophthalmic flow regulating implants which are used for the treatment of glaucoma.

### BACKGROUND OF THE INVENTION

Medical implants used to regulate the flow of fluids within the human body are known and used. One application for the use of such implants is in the treatment of glaucoma. Glaucoma is an eye condition characterized by an increase in the intraocular pressure (IOP) of the eye to an abnormal level. A normal eye maintains a proper IOP by the circulation within the eye of aqueous humor aqueous humor is secreted from the ciliary body, passes through the pupil into the anterior chamber of the eyeball, and is filtered out of the eyeball via the trabeculum and the Canal of Schlemm. With glaucoma, the aqueous humor excretory pathway is blocked, the aqueous humor cannot pass out of the eyeball at an adequate rate, the IOP rises, the eyeball becomes harder, and the optic nerve atrophies by the pressure applied on its fibers leaving the retina. A characteristic optic neuropathy develops, resulting in progressive death of the ganglion cells in the retina, restriction of the visual field, and eventual blindness. Advanced stages of the disease are characterized also by significant pain.

Glaucoma treatment, if initiated early in the course of the disease, can prevent further deterioration and preserve most of the ocular functions. The goal of glaucoma treatment is to reduce the IOP to a level which is considered safe for a particular eye, but which is not so low as to cause ocular malfunction or retinal complications.

One type of glaucoma treatment is filtration surgery, which provides an alternate route for aqueous humor to exit the anterior chamber of the eyeball and enter the sub-conjunctival space, thereby lowering the IOP. In full thickness operations a fistula is created through the limbal sclera, connecting directly the anterior chamber of the eyeball and the sub-conjunctival space. Full thickness operations provide long-lasting control of IOP; however, excessive loss of aqueous humor from the eyeball during the early postoperative period frequently leads to hypotony.

In guarded filtration surgery (trabeculectomy), a fistula created through the limbal sclera is protected by an overlying partial thickness sutured scleral flap. The scleral flap provides additional resistance to excessive loss of aqueous humor from the eyeball, thereby reducing the risk of early postoperative hypotony. However, trabeculectomy may result in higher eventual IOP and increased risk of late failure of filtration, compared with full thickness operations.

In accordance with one recently introduced procedure, a full thickness filtering fistula may be created by a holmium laser probe, with minimal surgically induced trauma. After retrobulbar anesthesia, a conjunctival incision (approximately 1 mm) is made about 12-15 mm posterior to the intended sclerostomy site, and a laser probe is advanced through the sub-conjunctival space to the limbus. Then, multiple laser pulses are applied until a full thickness fistula is created. This technique has sometimes resulted in early hypotony on account of a difficulty in controlling the sclerostomy size. In addition, early and late iris prolapse into the sclerostomy has resulted in abrupt closure of the fistula and eventual surgical failure. Further, despite its relative simplicity, the technique still necessitates the use of retrobulbar anesthesia to avoid pain caused by the laser applications. The injection of anesthetic material close to the already damaged optic nerve may sometimes lead to further visual damage. A further disadvantage of this procedure, as well as other types of glaucoma filtration surgery, is the propensity of the fistula to be sealed by scarring.

Various attempts have been made to overcome the problems of filtration surgery, for example, by using ophthalmic implant devices. Typical ophthalmic implants utilize drainage tubes so as to maintain the integrity of the openings formed in the eyeball for the relief of the IOP. The assignee of this invention is also the assignee of several other U.S. patents and patent applications directed to drainage implants, methods of implanting and using those implants, and delivery devices for delivering and implanting those implants. These include U.S. Pat. Nos. 5,702,414; 5,868,697; 5,968,058; and 6,203,513 and U.S. patent application Ser. Nos. 09/324,694; 09/383,472; and 09/729,050. The disclosures of these patents and patent applications are hereby expressly incorporated by reference as if fully set forth herein. The delivery devices described and depicted herein may be used with the implants as described and depicted in those patents and patent applications.

In U.S. Pat. No. 6,203,513, FIG. 28 illustrates a distal end portion of a delivery device 110. The delivery device 110 has a handle (not shown) and a rodlike instrument in the form of a tube 112. The tube 112 has a central bore 114 in which is located a retractable wire 116. The retractable wire 116 is positioned for penetrating a tube passage 102 of the implant 100 when the implant 100 is attached to the delivery device 110. The delivery device 110 has a retention mechanism including an abutment surface 118 having an angle generally corresponding to that of a flange or disk 106 of the implant 100, for preventing the implant 100 from moving up the delivery device 110 during implantation, and a hook 120 for preventing the implant 100 from moving down the wire 116. The angled abutment surface 118 also prevents the implant 100 from rotating relative to the delivery device 110.

For implantation, the implant 100 is placed over the wire 116 with the wire 116 projecting into the tube passage 102 and with the abutment surface 118 abutting against the flange or disk 106, with the hook 120 retaining the flange or disk 106 around the opposite side. FIG. 28 illustrates the distal end of the delivery device 110 in this condition, with the retention wire 116 in its forward position.

After the implant is in position, the retention wire 116 is retracted out of the implant 100. FIG. 29 of U.S. Pat. No. 6,203,513 illustrates the end of the delivery device 110 with the retention wire 116 retracted. With the retention wire 116 retracted, the implant 100 is free to slide away from the hook 120, allowing the delivery device 110 to be withdrawn, leaving the implant 100 in place.

### SUMMARY OF THE INVENTION

It is an object of the invention to provide improved delivery devices for delivering and/or implanting implants used to regulate the flow of fluids within the body. The delivery devices may be used, for example, to deliver and implant ophthalmic implants, which may be implanted into the eyeball for the treatment of glaucoma.

An example of a delivery device within the scope of the invention comprises a handle and a tube (such as a hollow needle or cannula), with a retractable wire extending through the tube. Prior to deployment, the wire extends a short distance out of the distal end of the tube. The drainage implant is mounted on the wire at the distal end of the tube; for example, the implant may have a central bore into which the wire extends. The implant may be held on the wire by friction, by a hook, or by other suitable means. The delivery device includes a trigger that acts on the retractable wire. Actuating the trigger retracts the wire into the tube, thereby releasing the implant.

With such a construction, the physician can easily deliver the implant to the appropriate site. When implant is in place, pressing the trigger releases the implant from the delivery device such that the implant can be left in place and the delivery device can be withdrawn.

Various features may be incorporated into the delivery devices according to the invention. For example, the handle may have a holding area that includes notches designed for improving grip. In addition, the holding area may have a triangular or other suitable cross-sectional profile that provides the user with the ability to control the orientation of the delivery device as the implant is inserted.

The handle may be similar in length to other tools employed by ophthalmologists. In addition, the handle may be designed such that the user can operate the device with either hand.

The distal end of the tube may be configured for cooperation with the implant. For example, the distal end of the tube may be angled to match an angled disk or flange on the implant. In this manner, the implant in prevented from rotating relative to the delivery device. Because the implant is prevented from rotating on the delivery device, the physician is able to know the position of the implant from knowing the position of the delivery device. For example, the implant may be oriented on the delivery device such that the implant is in the correct position for implantation if the trigger is facing away from the eye, or if the trigger is facing up.

The wire may extend all the way through the tube of the delivery device. The proximal end of the wire may be adhered or otherwise rigidly connected to the handle. The trigger may act on a portion of the wire that is between the proximal end of the tube and the area where the wire is adhered or connected to the handle.

Features may be incorporated to prevent undesired actuation of the trigger. For example, a removable safety wedge may be used to prevent movement of the wire until the wedge is removed. In addition, a suitable distance may be provided between the holding area and the trigger, such that the delivery device may be maneuvered simply by holding the holding area, and not the trigger.

Persons skilled in the art will appreciate that delivery devices according to the invention may be designed to be disposable and incapable of multiple uses.

An example of a method for implanting an ophthalmic implant with a delivery device according to the invention may start with cutting a small slit in a portion of the conjunctiva which normally lies at a distance away from the intended implantation site. As the implant itself is very small, the slit also may be very small. The small size of the slit, as well as its positioning at a distance away from the implantation site, helps prevent contamination of the sclerostomy site and reduces the risk of infection.

The physician may then use the delivery device to place the implant through the slit, to direct it to the implantation site, and to insert it into the sclera at the implantation site. The sclera may be pierced by the tip of the implant.

Once the implant is in the desired position in the eye, the physician may then press the trigger to retract the wire and release the implant. The action of the trigger may occur in three stages, which allows the user to control the timing and the release rate of the implant. In this example, the initial movement is an idle movement that permits the user to push the trigger down easily. The next stage moves the wire through its elastic strain range and slightly retracts the wire from the implant, thereby beginning the releasing process. Finally, in the third stage the wire is forced into a notch in the handle, fully retracting the wire into the tube and fully releasing the implant.

An implant delivery device according to the invention has other applications aside from the field of intraocular implants. For example, the implant delivery device may be used to delivery and insert an implant for drainage of a hydrocele sac, regulating flow between the hydrocele sac and the subcutaneous scrotum. As will be appreciated by persons of ordinary skill in the art, other applications in accordance with the invention are possible.

### BRIEF DESCRIPTION OF THE DRAWINGS

FIG. 1 is an isometric view of a delivery device according to the invention;

FIG. 2 is a side view of the delivery device of FIG. 1;

FIG. 2A is an illustrative cross-sectional view, taken along the line A-A in FIG. 2, of the holding area of the handle of the delivery device shown in FIG. 1; and

FIGS. 3A through 3D illustrate the actuation of the trigger of the delivery device of FIG. 1, with the consequent retraction of the wire.

### DETAILED DESCRIPTION OF THE DRAWINGS

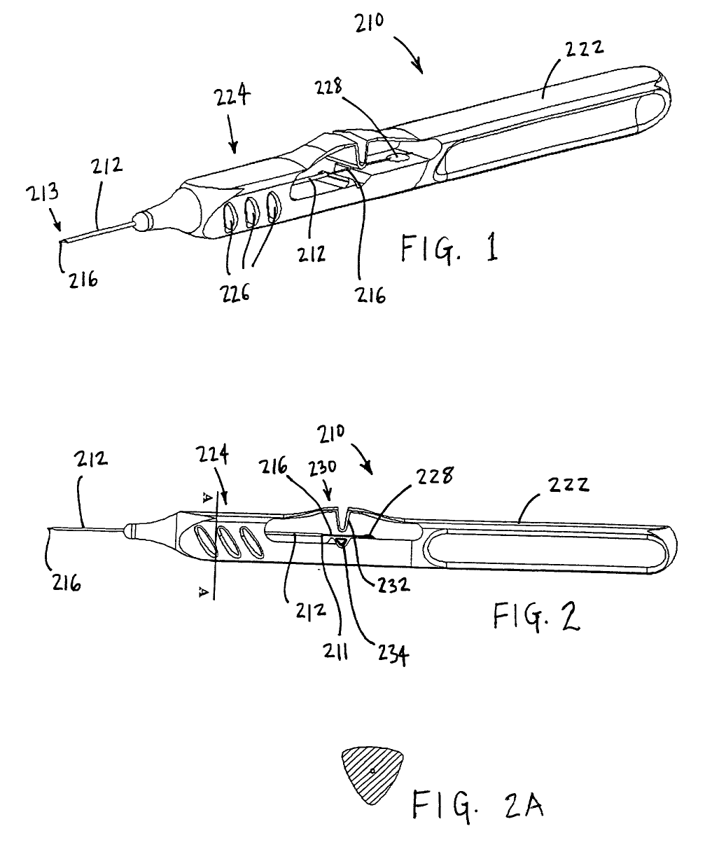

FIGS. 1 and 2 illustrate isometric and side views, respectively, of an example of a delivery device 210. The delivery device 210 comprises a handle 222 and a tube 212, with a retractable wire 216 extending through the tube 212. Prior to deployment, the wire 216 extends a short distance out of the distal end 213 of the tube 212. As illustrated in FIGS. 28 and 29 of U.S. Pat. No. 6,203,513, a drainage implant may be mounted on the wire 216 at the distal end 213 of the tube 212.

As in the delivery device illustrated in FIGS. 28 and 29 of U.S. Pat. No. 6,203,513, the distal end 213 of the tube 212 has an abutment surface that is angled to match an angled flange or disk on the implant. In this manner, the implant in prevented from rotating relative to the delivery device 210. The abutment surface also prevents the implant from moving up the delivery device 210 during implantation. In this embodiment, the implant is held on the wire 216 by a friction fit, which prevents the implant from moving down the wire 216. This embodiment does not have a hook, although a hook may alternatively be used, as in FIGS. 28 and 29 of U.S. Pat. No. 6,203,513. With the friction fit, the hook is optional.

The handle 222 has a holding area 224 that includes notches 226 designed for improving grip. As shown in FIG. 2A, the holding area 224 has a generally triangular cross-sectional profile, which provides the physician with the ability to control the orientation of the delivery device 210, and thus the orientation of the implant.

In this illustrated embodiment, the wire 216 extends all the way through the tube 212. The proximal end of the wire 216 may be adhered or otherwise rigidly connected to the handle, for example at wire attachment area 228.

The delivery device 210 includes a trigger 230 that acts on the retractable wire 216. Pressing the trigger 230 retracts the wire 216 into the tube 212, thereby releasing the implant. In the illustrated embodiment, the trigger 230 is configured as a resilient spring, biased in the non-actuated position as shown in FIGS. 1 and 2. Pressing the trigger 230 causes a contact portion 232 of the trigger 230 to engage a portion of the wire 216 that is between the proximal end 211 of the tube 212 and the wire attachment area 228, where the wire is adhered or connected to the handle 222. As shown in FIG. 2, a removable safety wedge 234 may be used to prevent movement of the wire 216 until the wedge 234 is removed. The wedge 234 may be removed just prior to use of the delivery device 210, or just prior to actuation of the trigger 230.

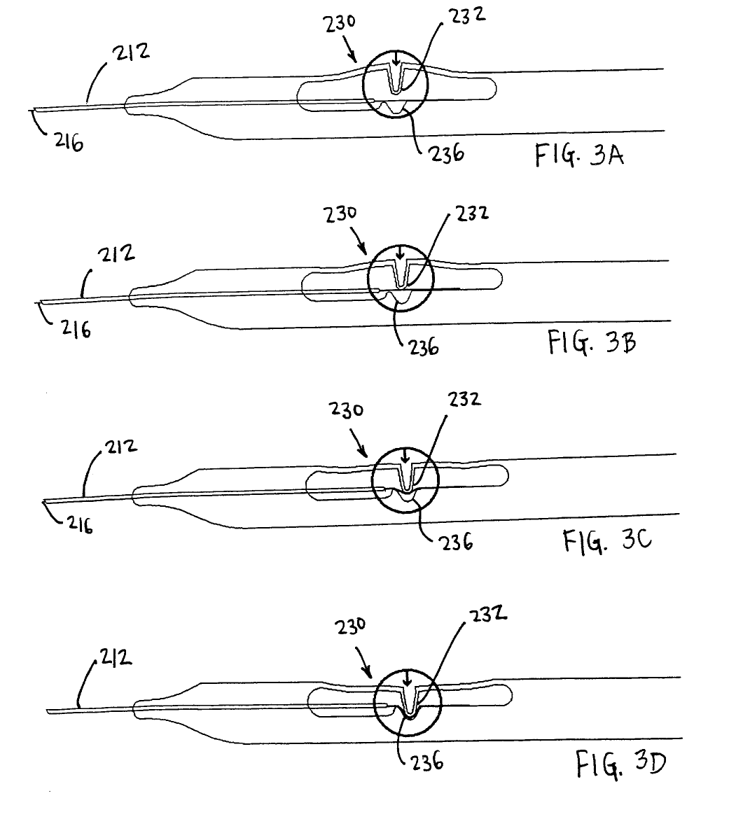

Once the implant is in the desired implantation position, the physician may then press the trigger 230 to retract the wire 216 and release the implant. The action of the trigger 230 may occur in three stages, which allows the user to control the timing and the release rate of the implant. In this example, the initial movement occurs between the relaxed position shown in FIG. 3A and the position shown in FIG. 3B. This initial movement is an idle movement that permits the user to push the trigger 230 down easily, without movement of the wire 216. In this manner, the physician can control the proper pressure on the trigger, which allows the physician to control the distance to which the trigger is depressed. The next stage, which occurs from the position shown in FIG. 3B to the position shown in FIG. 3C, moves the wire 216 through its elastic strain range and slightly retracts the wire 216 from the implant, thereby beginning the releasing process. Finally, in the third stage, which occurs from the position shown in FIG. 3C to the position shown in FIG. 3D, the wire 216 is forced into a notch 236 in the handle 222, fully retracting the wire 216 into the tube 212 and fully releasing the implant. The end of the wire 216 may be retracted into the tube 212 further than the distal end 213 of the tube, to insure that the wire 216 will not come out of the tube 212 when the trigger 230 is released.

The retractable wire 116 in a delivery device such as that illustrated in FIGS. 28 and 29 of U.S. Pat. No. 6,203,513 may be retracted by action of an actuating mechanism or trigger in accordance with the present invention. The actuating mechanism or trigger may take any suitable form, for example as shown in the embodiment of FIGS. 1 through 3.

As will be appreciated by persons having ordinary skill in the art, the various embodiments of delivery devices described above are given by way of example only. Various changes, modifications and variations may be applied to the described embodiments without departing from the scope of the invention, defined by the appended claims.

What is claimed is:

1. A delivery device for implanting an implant, the delivery device comprising:
a handle;
a rodlike instrument having a bore;
a retractable wire located in the bore of the rodlike instrument;
a retention mechanism including an abutment surface for preventing the implant from moving up the delivery device during implantation and means for preventing the implant from moving down the wire during implantation; and
a trigger for retracting the retractable wire.

2. A delivery device according to claim 1 wherein the means for preventing the implant from moving down the wire during implantation comprises a hook, and wherein the hook prevents movement of the implant in a direction parallel to the wire but permits movement in a direction transverse to the wire, such that when the wire is retracted from a tube passage of the implant, the implant is permitted to slide away from the hook to separate the implant from the delivery device.

3. A delivery device according to claim 1 wherein the abutment surface has an angle generally corresponding to that of a disk of the implant.

4. A delivery device for implanting an implant, the delivery device comprising:
a handle;
a tube having a bore;
a retractable wire located in the bore of the tube; and
a trigger for retracting the retractable wire.

5. A delivery device according to claim 4, wherein the delivery device further comprises means for retaining the implant on the delivery device.

6. A delivery device according to claim 5, wherein the means for retaining the implant on the delivery device includes an abutment surface for preventing the implant from moving up the delivery device during implantation.

7. A delivery device according to claim 6 wherein the abutment surface has an angle generally corresponding to that of a disk of the implant.

8. A delivery device according to claim 5, wherein the means for retaining the implant on the delivery device includes a hook for preventing the implant from moving down the wire during implantation.

9. A delivery device according to claim 5, wherein the means for retaining the implant on the delivery device includes a friction fit between the wire and the implant for preventing the implant from moving down the wire during implantation.

10. A delivery device according to claim 4, wherein, prior to deployment, the wire extends a short distance out of a distal end of the tube.

11. A delivery device according to claim 10, wherein actuating the trigger retracts a distal end of the wire fully within the tube.

12. A delivery device according to claim 4, wherein the handle has a holding area that includes notches for improving grip.

13. A delivery device according to claim 4, wherein the handle has a generally triangular cross-sectional profile.

14. A delivery device according to claim 4, wherein, prior to deployment, the wire extends all the way through the tube of the delivery device.

15. A delivery device according to claim 14, wherein a proximal end of the wire is adhered to the handle, and wherein the trigger acts on a portion of the wire that is between a proximal end of the tube and an area where the wire is adhered to the handle.

16. A delivery device according to claim 4, further comprising a removable safety wedge to prevent movement of the wire until the wedge is removed.

17. A delivery device according to claim 4, wherein actuation of the trigger presses a portion of the wire into a notch formed in the handle.

18. A delivery device according to claim 4, wherein the trigger is adapted to be pressed a short distance before it engages the wire.
